(19) United States
(12) Patent Application Publication
(10) Pub. No.: US 2018/0116586 A1
(43) Pub. Date: May 3, 2018

(54) Methods and Apparatus For Providing A Breath-Hold Feedback Signal To A Patient During Respiration

(71) Applicant: PrecisionRad LLC, Scottsdale, AZ (US)

(72) Inventor: Colin Thompson, Scottsdale, AZ (US)

(21) Appl. No.: 15/337,622

(22) Filed: Oct. 28, 2016

(51) Int. Cl. A61B 5/00; A61B 5/113; A61B 6/03

(52) U.S. Cl. CPC A61B 5/486; A61B 5/1135; A61B 5/6823; A61B 5/7278; A61B 5/742; A61B 2562/0223; A61B 5/7405; A61B 6/032; A61B 2562/0219; A61B 2562/0261; A61B 5/7455

(57) ABSTRACT

Systems, devices, and methods for providing a feedback signal to assist a patient in repeating a breath-hold at the same point in successive respiratory cycles. The tracking device includes: a spring-biased wheel; a strap configured to wrap around a patient and having a segment configured to wind around a portion of the wheel when the patient exhales, and to unwind when the patient inhales; and a processor configured to detect an angular position of the wheel and to present the patient with a corresponding feedback signal.

### TECHNICAL FIELD

The present invention relates, generally, to a strap-on transducer for providing a feedback signal indicative of thoracic movement and, more particularly, to a spring biased wheel about which the strap is wound and unwound to indicate a breath-hold set point.

### BACKGROUND

Computed tomography (CT) is a common imaging modality used for both medical diagnostics and imaging guidance for minimally invasive procedures including but not limited to biopsies, drainage catheter placement, and ablation. All of these procedures require precise placement of a surgical instrument at some predetermined location within the body. Intermittent CT scanning is performed to check the location of the instrument as it is incrementally advanced along a predetermined trajectory to the target lesion. One of the difficulties encountered while performing these procedures surrounds accurately targeting locations within the body subject to significant respiratory motion induced by the diaphragm, such as the lungs and upper abdominal organs. The location of lesions within these organs can change significantly throughout the respiratory cycle.

Interventional radiology is a subspecialty of radiology that specializes in minimally invasive image-guided procedures. The ability to consistently reproduce a breath hold at the same point in the respiratory cycle and, hence, at the same lung volume, is difficult even for fully conscious patients with normal respiratory function. Failure to perform a breath hold at the same point within successive respiratory cycles can lead to inaccurate placement of surgical instruments which prolongs procedure time, increases radiation dose, increases risk of organ injury, and increases risk of failed procedures e.g. non-diagnostic biopsies. Therefore, the ability to monitor the respiratory cycle and provide feedback to the patient to assist the patient in consistently holding their breath at a specific lung volume could minimize these problems while performing procedures under CT guidance.

Breath-hold monitoring systems which provide feedback to help a patient hold his/her breath at a particular position are generally well known. See, for example, Felmlee et al. U.S. Pat. No. 7,678,063, which discloses a motion detection and monitoring system including an expandable bellows connected to a belt. The bellows respond to expanding and contracting chest motion. A hollow tube is connected to the interior of the bellows. Changes in patient girth during respiration results in expansion and contraction of the tubing. A pressure transducer communicates with lit diodes on a display representing displacement of the detected patient motion from a reference position. The motion detection system is calibrated so that a lit diode represents a unit of displacement. Thus, the patient is able to use the display as feedback during an examination to reproduce a desired respiratory level during treatment.

Other presently known systems similarly attempt to linearly correlate body movement to pressure changes within a gas pressure transducer, often leading to inaccurate results due to, inter alia, hysteresis effects inherent in the pressure transducer.

Improved systems and methods for reliably providing visual feedback to facilitate repeatable breath-holds are thus needed which overcome the foregoing limitations.

### BRIEF SUMMARY OF THE INVENTION

The present invention relates to an improved respiratory tracking device including a spring-biased shaft about which a wearable strap winds and unwinds during respiration. A sensor such as an accelerometer, rotary encoder, gyrometer, magnetometer, strain gauge, or slotted wheel cooperates with the shaft and a processor to establish a breath-hold set point, and to provide visual, audible, and/or haptic feedback indicative of the set point during subsequent respiratory cycles.

Various other embodiments, aspects and features of the present invention are described in more detail below. Additional features and characteristics will become apparent from the subsequent detailed description and the appended claims, taken in conjunction with the accompanying drawings and this background section.

### BRIEF DESCRIPTION OF THE DRAWING FIGURES

Exemplary embodiments will hereinafter be described in conjunction with the following drawing figures, wherein like numerals denote like elements, and:

FIG. 1 is a schematic perspective view of internal components of a respiratory tracking device in accordance with various embodiments;

FIG. 2 is a schematic perspective view of the components of FIG. 1 disposed within an integrated housing in accordance with various embodiments;

FIG. 3 is an alternate schematic perspective view of internal components of a respiratory tracking device in accordance with various embodiments;

FIG. 4 a schematic perspective view of the components of FIG. 3 disposed within an integrated housing in accordance with various embodiments; and

FIG. 5 is a schematic perspective view of a slotted wheel module for tracking the angular position of a breath-hold transducer in accordance with various embodiments.

### DETAILED DESCRIPTION OF PREFERRED EXEMPLARY EMBODIMENTS

The following detailed description is merely exemplary in nature and is not intended to limit the invention or the application and uses of the invention. Furthermore, there is no intention to be bound by any theory presented in the preceding background or the following detailed description.

The present invention is a noninvasive respiratory monitoring device which provides a quantifiable signal representative of the lung volume. The quantifiable signal is used to provide feedback to the operator and patient to assist the patient in consistently reproducing breath holds at the same lung volume.

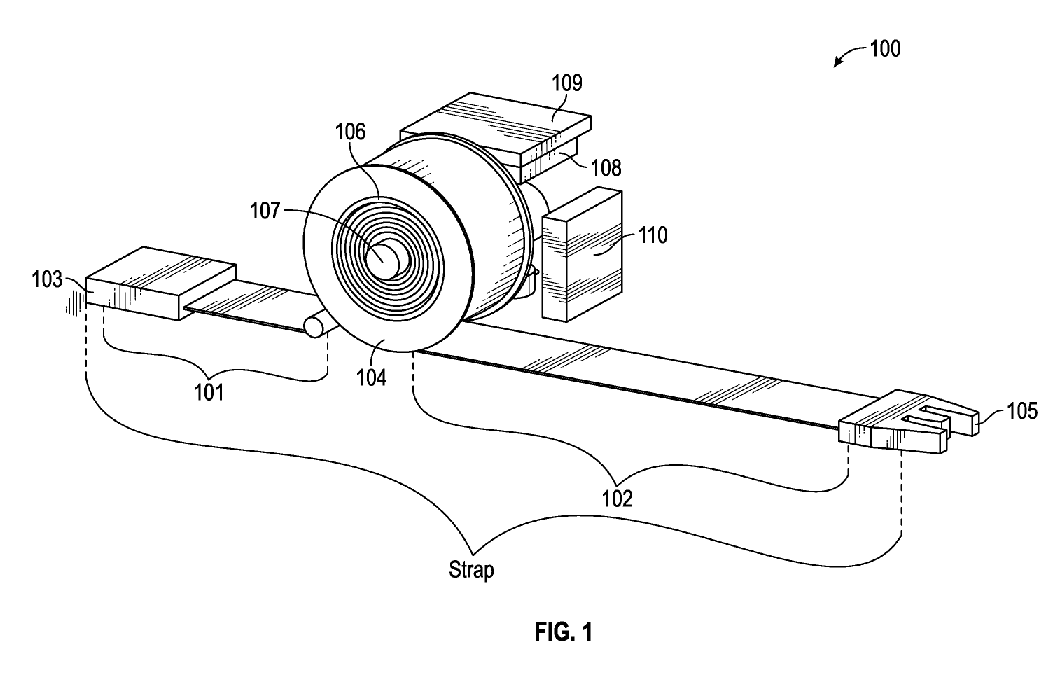

A first embodiment of the invention consists of an accelerometer, microcontroller, display, sensory feedback mechanism, battery, slip ring, ball bearings, clock spring (aka power spring), strap, wheel around which the strap is wrapped, and external housing (FIGS. 1a-d). All of the components are contained within the small external housing. Analogous to many other inventions such as retractable dog leashes, tape measures, power cords, etc., a portion of the strap is wrapped around a wheel within the housing. The clock spring is connected to both the wheel and the housing and serves to retract the strap when the strap is unwound from the wheel. During patient respiratory inspiration (the patient inhales), the expanding chest/abdominal cavity pulls the strap so that it partially unwinds from the wheel. This unwinds several centimeters of the strap from the wheel to accommodate the increased chest/abdominal circumference. During patient respiratory expiration (patient exhales), the several centimeters of the strap retract back around the wheel under the action of the clock spring. The process is repeated for successive respiratory cycles.

As the strap is unwound and rewound around the wheel, the wheel is rotated by an angular distance which is proportional to the circumference changes of the chest/abdomen. Ball bearings may be placed between the wheel and the portion of the housing that connects to the wheel to allow low friction movement between the two as the patient breathes. The accelerometer is attached to the wheel so that it rotates back and forth every respiratory cycle as well. The amount of rotation of the wheel is detected by the accelerometer. The accelerometer is connected to a microcontroller via wires as part of a small slip ring so that the accelerometer can rotate while maintaining electrical connectivity to the microcontroller.

The data collected by the accelerometer is processed by the microcontroller to yield degrees of angular rotation. Consequently, any specific point in the respiratory cycle may be represented as a degree of rotation of the accelerometer. The microcontroller can also analyze the accelerometer data to determine when the patient is holding their breath. This can be achieved, for example, by calculating the standard deviation of the incoming accelerometer data. When the standard deviation approaches zero for a short period of time, this indicates the patient is holding their breath because the accelerometer is not moving. This value represents the specific point in the respiratory cycle at which the patient held their breath and is recorded into memory by the microcontroller.

In an embodiment, the processed accelerometer data may be displayed on a small screen attached to the accelerometer housing. A sensory feedback mechanism such as a small vibration motor (analogous to the haptic transducer that causes a cell phone to vibrate) within the housing can be activated when the recorded breath hold value is achieved to serve as additional feedback to a patient. This assists a patient in consistently reproducing a breath hold at the desired lung volume. Although one embodiment provides haptic feedback, i.e. a motor that vibrates in response to applied voltage, any combination of other forms of visual, auditory, or other sensory feedback could be employed. A user interface may include a touch screen or buttons on the screen allowing the operator to set the recorded breath hold value to provide sensory feedback as described above.

In alternative embodiments, the display and user interface may be separate from the portion of the device strapped to the patient. In these embodiments, the display may be placed proximate the operator and/or patient allowing the patient to see the display for visual feedback to assist in reproducing consistent breath holds. This can be achieved, for example, by attaching the display to the CT scanner, a separate stand, or to the patient and/or operator as a wearable display. The visual feedback depicted on the display may include any form of numerical and/or graphical representations of the signal representing the respiration cycle. The processed data from the device strapped to the patient may be transmitted through wires or wirelessly to the display (e.g., Bluetooth, WiFi, GPS).

In a further embodiment, the display may be a component of another medical device which receives the data via wired or wireless communication. For example, the other medical device may be one used for guidance during CT guided procedures. A specific example of such a guidance device is disclosed in U.S. patent application Ser. No. 15/097,054, the entire contents of which are hereby incorporated herein by this reference. These devices are used to assist the placement of a surgical instrument at a desired location in the patient's body. Augmenting a guidance device with the ability to control respiratory variability could enhance the safety and accuracy of CT guided procedures.

In additional embodiments, a quantifiable signal representative of lung volume may be obtained using sensors other than a rotating accelerometer. Any sensor capable of detecting rotational or translational motion could be used in place of the accelerometer. Examples of other sensors include rotary encoders, gyrometers, and magnetometers.

More particularly, rotary encoders are a class of sensors used to detect angular position or rotational motion of a shaft, axle, or wheel. Rotary encoders are sometimes classified as absolute or incremental, depending on whether they give an absolute angular position or a position relative to a reference position. Rotary encoders can use conductive, capacitive, optical, and/or magnetic methods of detecting rotation, and generate an output signal representative of rotational motion and/or position. The details of the inner workings of rotary encoders is well described elsewhere and is beyond the scope of this document. The rotary encoder may be attached to the rotating wheel or shaft of the wheel and outputs a signal indicating its angular position throughout the respiratory cycle used in the same manner previously described for the accelerometer.

In other embodiments, microelectromechanical systems (MEMS) devices employing gyrometers and/or magnetometers may be employed in lieu of (or in addition to) an accelerometer to detect position and/or motion. Gyrometers detect angular motion and magnetometers detect position or orientation relative to the Earth's magnetic field. Gyrometers output angular velocity. The angular position can then be determined by calculating the integral of angular velocity with respect to time. Use of the magnetometer would differ from the accelerometer in that the wheel would be oriented orthogonal relative to the orientation used with the accelerometer so that a rotation in the wheel rotates the magnetometer in a plane perpendicular to gravity.

Further examples of sensors which could be used to measure changes in chest/abdominal circumference include strain gauges and piezoelectric elements. Both of these types of sensors would be used to measure the tension on a strap wrapped around the patient. As the patient breathes the tension varies with the changes in chest/abdominal circumference which is represented by changes in voltage output. In the case of a strain gauge, resistance across the strain gauge changes with varying amounts of tension which changes the voltage output. A piezoelectric element would similarly output a voltage proportional to the pressure or strain on the piezoelectric element occasioned by the respiratory cycle. An amplifier may be employed to better detect and discriminate small changes in chest/abdominal circumference. The wheel, clock spring, and slip ring would be unnecessary in these embodiments. (See, for example, U.S. Pat. Nos. 5,543,012 A and 6,561,987 B2, the entire contents of which are hereby incorporated herein by this reference).

A general approach to using the device for a CT guided interventional procedure may be described as follows: The device is attached around the chest or upper abdomen of a patient. An initial CT scan is performed with the patient holding their breath to plan the desired percutaneous trajectory to the lesion or other location of interest. Once a path is identified on the CT images, a marker is placed on the patient's skin at the skin entry point for the surgical instrument. The value corresponding to the patient's lung volume at which a breath-hold was performed during the scan was automatically recorded by the microcontroller as mentioned above. This value can then be set as the desired breath-hold reference value by the push of a button on the screen. Thereafter, whenever the patient achieves the lung volume corresponding to that set value, the vibrating motor activates and the patient feels the vibration (or sees the display indicate the desired level). The patient can then consistently hold their breath at the lung volume that activates the motor and/or achieves the desired visual feedback. Subsequent scans are then performed intermittently as needed to place the surgical instrument along the desired trajectory to the lesion of interest.

During each scan and during advancement of the surgical instrument, the patient is instructed to hold their breath at the lung volume that has been set to activate the vibrating motor and/or achieves the desired visual feedback. In this manner, changes in the location of the targeted lesion due to variations in lung volumes during breath holding are mitigated. Thus the desired trajectory as identified on the initial planning CT scan remains essentially unchanged throughout the procedure.

In the current preferred embodiment, the intended use of the device is to improve accuracy of surgical instrument placement during CT-guided interventional procedures. There is, however, a potentially broader range of applications. One such application is to improve efficacy of external beam radiation therapy. By minimizing variability in lung volume during repeated breath holding, the location of a tumor subject to respiratory motion is held constant from one breath hold to the next. This allows a radiation dose to be delivered more precisely to the intended target such as a lung cancer. This will increase the radiation dose to the tumor and decrease radiation dose to surrounding normal tissues.

Another application of the devices described herein surrounds the detection of apneic episodes. The device detects when a patient is not breathing and can record the duration, time of occurrence, and number of episodes. For example, this could play a role in diagnosing sleep apnea, detecting apnea of prematurity in the neonatal intensive care unit, or detecting respiratory arrest in hospitalized patients. Furthermore the invention could assist in the treatment or prevention of prolonged apnea by stimulating the patient to breathe with sensory feedback.

The above embodiments contemplate a discrete transition to haptic feedback when a breath-hold reaches a predetermined set point. Alternatively, the haptic could be more gradual, with the vibrational amplitude increasing as the set point is approached, reaching a maximum value at the set point. In this way, the patient “feels” the feedback to a greater degree as the set point approaches, allowing more precise control over the point in the respiratory cycle at which the breath-hold occurs.

Alternatively, visual feedback could be used as a sole feedback mechanism or in addition to the haptic feedback. For example a row of light emitting diodes (LEDs) or a bar graph on a display may indicate points in the respiratory cycle. The LEDs or graph may sequentially illuminate and/or vary in color or intensity during inspiration and expiration towards a specific LED or point on the graph indicative of the predetermined set point. When the patient reaches the predetermined set point, the specific LED or point on the graph indicating the set point will be illuminated. A simultaneous haptic feedback may be applied to further facilitate a sedated patient in maintaining the desired lung volume. Another example of visual feedback is a circular gauge analogous to a clock face with one hand indicative of the current position in the respiratory cycle and another fixed reference indicative of the set point. The patient can then align the moving hand with the fixed reference by inhaling or exhaling to the desired lung volume. Again, maintaining alignment can be facilitated by haptic feedback. The above examples are just a few possibilities among countless other forms of graphical and/or numerical representations of the data. These are for illustrative purposes and are not intended to limit the scope of the invention.

Referring now to FIG. 1, a tracking device 100 includes a first strap portion 101 having a first distal connector 103 and a second strap portion 102 having a second distal connector 105 configured to be releasably secured to the first connector 103. The strap portions 101, 102 are configured to wrap around a patient's chest or abdominal area, with the ends 101, 103 secured together such that the second strap portion 102 extends during inspiration and retracts during expiration, as described more fully below.

The device further includes a wheel 104 configured to rotate about a shaft 107, and a spring connecting the wheel 104 to the shaft 107. The second strap portion 102 wraps around the wheel 104 when the wheel rotates in the clockwise direction, and unwraps when the wheel rotates counterclockwise. In this way, the spring-loaded wheel can allow the length of the second strap portion 103 to extend when the patient inhales, and to take up the slack when the patient exhales. A control circuit 108 is configured to convert the angular position of the wheel to a graphical and/or numeric display on a screen 109. A battery 110 provides power to the various components.

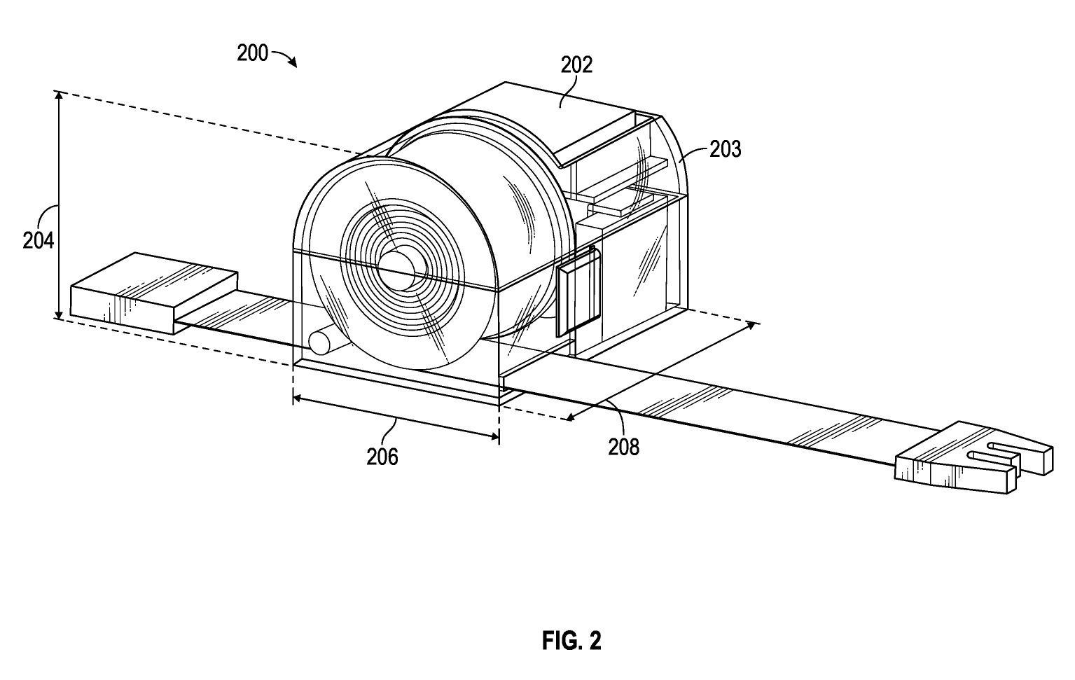

FIG. 2 shows the components of FIG. 1 disposed within a housing. In particular, a tracking device 200 includes a display screen 202 integrated into a surface of a housing 203 characterized by a height dimension 204, a width dimension 206, and a length dimension 208. In an embodiment, the height dimension 204 is in the range of about 20 to 100 millimeters (mm), and preferably about 40 mm; the width dimension 206 is in the range of about 10 to 100 millimeters (mm), and preferably about 40 mm; and the length dimension 208 is in the range of about 20 to 150 millimeters (mm), and preferably about 60 mm.

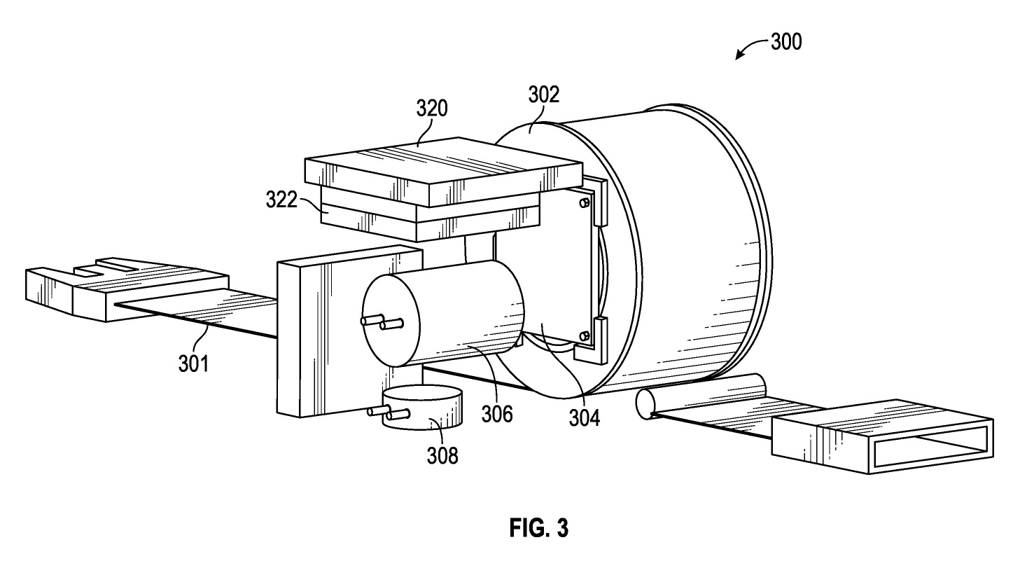

FIG. 3 is an alternate view of internal components of a respiratory tracking device 300 including a strap 301 wound around a wheel 302, a display screen 320, a microcontroller 322, an angular position detector (e.g., accelerometer) 304, a slip ring 306, and a sensory feedback module (e.g., a haptic device such as a vibrating motor) 308. More particularly, the accelerometer monitors the angular position of the wheel as the patient breathes in and out, and provides a signal to the microcontroller representative of the then-current angular position of the wheel. When the patient performs a breath hold, the accelerometer signal stabilizes (e.g., remains constant), causing the microcontroller to “save” the angular position of the wheel in memory. Alternatively, a strain gauge (not shown) or other suitable mechanism may be used to determine the angular position (or angular travel) of the wheel from a reference point. During subsequent respiratory cycles, the display screen 320 and/or the haptic device 308 provides an indication to the patient as the saved position is approached, allowing the patient to perform a subsequent breath-hold at the same saved point based on the visual, audio, and/or haptic feedback.

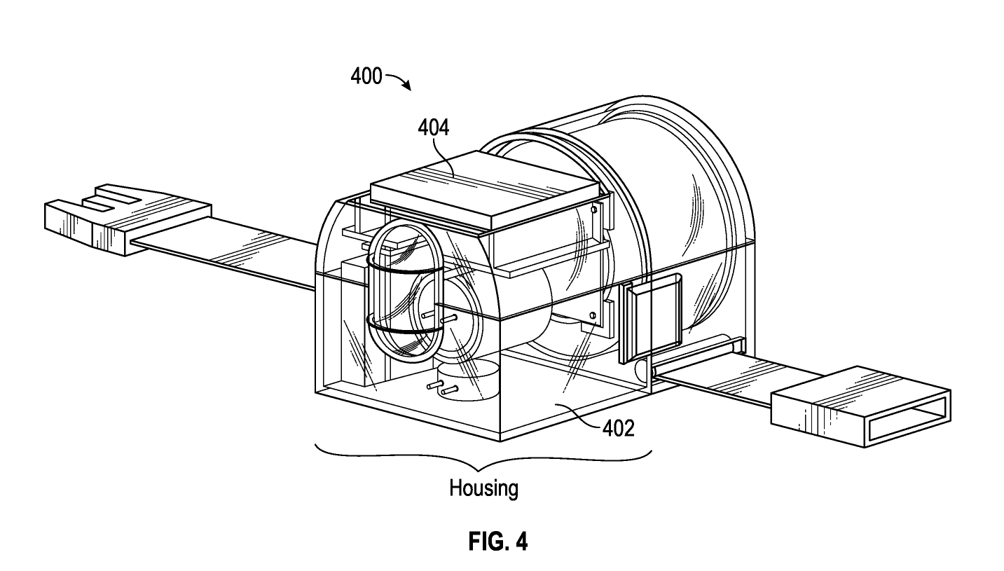

FIG. 4 shows a tracking device 400 including a display screen 404 integrated into a housing 402.

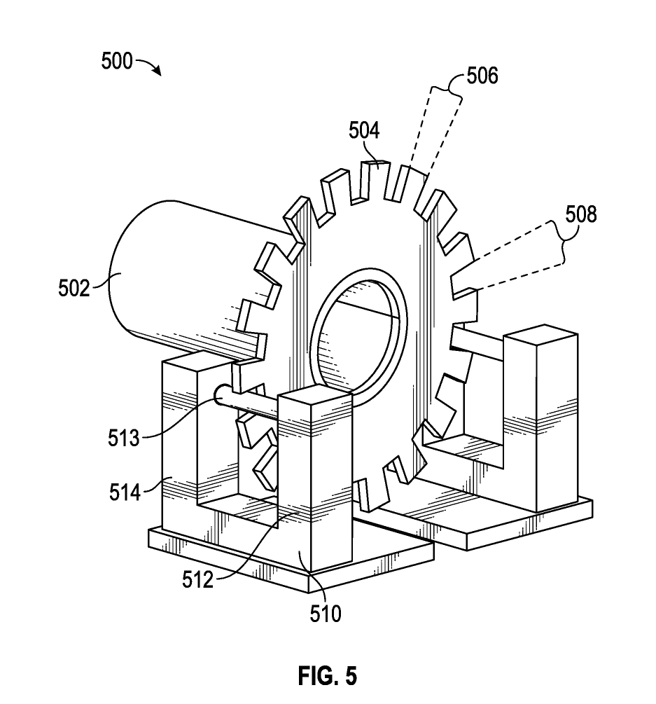

Referring now to FIG. 5, an alternative implementation of a tracking module 500 includes a winding shaft 502 about which a strap (not shown) is wound and unwound during successive respiratory cycles, a slotted wheel 504 configured to rotate with the shaft 502, and one or more photo detection module 510, each including a light emitter 512 configured to transmit light (e.g., infrared) 513 to an associated light detector 514. More particularly, the slotted wheel includes a plurality of alternating teeth 506 and slots 508.

With continued reference to FIG. 5, as the shaft 502 rotates during inspiration and expiration against a biasing mechanism (e.g., spring) (not shown in FIG. 5), the slots and teeth alternately permit and prevent passage of the light beam 513 to the detector 514. An associated processing circuit or chip (not shown in FIG. 5) tracks the angular position of the wheel 504 by counting the number of teeth/slots which pass by the light beam. This count is proportional to the degree of expansion and contraction of the chest or abdomen to which the device is attached, allowing the module 500 to precisely track and store breath-hold events.

A respiratory tracking device is thus provided which includes: a spring-biased wheel; a strap configured to wrap around a patient and having a segment configured to wind around a portion of the wheel when the patient exhales, and to unwind when the patient inhales; and a processor configured to detect an angular position of the wheel and to present the patient with a corresponding feedback signal.

In an embodiment, the device further includes a transducer configured to convert the angular position of the wheel into the feedback signal.

In an embodiment, the transducer comprises one or more of an accelerometer, strain gauge, slotted wheel, rotary encoder, gyrometer, and magnetometer.

In an embodiment, the device may include an emitter and a detector configured to pass a beam through the slotted wheel.

In an embodiment, the processor is configured to track successive interruptions in the beam.

In an embodiment, the feedback signal comprises a visual display.

In an embodiment, the feedback signal comprises a haptic signal.

In an embodiment, the feedback signal comprises a visual signal combined with one of a haptic signal and an audio signal.

A method is also provided for facilitating a breath-hold event at a predetermined point within a respiratory cycle. The method includes the steps of: providing a strap configured to wrap around a patient and having a segment configured to wind around a portion of a spring-biased wheel when the patient exhales, and to unwind when the patient inhales; providing a processor configured to detect an angular position of the wheel; and presenting the patient with a feedback signal corresponding to the angular wheel position.

In an embodiment, the method further includes converting the angular position of the wheel into the feedback signal using a transducer.

In an embodiment, the transducer comprises an accelerometer.

In an embodiment, transducer comprises a strain gauge.

In an embodiment, the transducer comprises a slotted wheel.

In an embodiment, the transducer further comprises an emitter and a detector configured to pass a beam through the slotted wheel.

In an embodiment, the method further includes tracking successive interruptions in the beam.

In an embodiment, presenting the feedback signal comprises displaying at least one of a graphical, audible, and haptic indication of a breath-hold set point.

In an embodiment, the feedback signal increases as the set point is approached.

A respiratory set point tracking device is also provided, the device including: a belt configured to extend around a patient and engage a spring-biased wheel; and a transducer configured to convert an angular position of the wheel into a haptic feedback signal.

As used herein, the word “exemplary” means “serving as an example, instance, or illustration.” Any implementation described herein as “exemplary” is not necessarily to be construed as preferred or advantageous over other implementations, nor is it intended to be construed as a model that must be literally duplicated.

While the foregoing detailed description will provide those skilled in the art with a convenient road map for implementing various embodiments of the invention, it should be appreciated that the particular embodiments described above are only examples, and are not intended to limit the scope, applicability, or configuration of the invention in any way. To the contrary, various changes may be made in the function and arrangement of elements described without departing from the scope of the invention.

What is claimed:

1. A respiratory tracking device comprising:
a spring-biased wheel;
a strap configured to wrap around a patient and having a segment configured to wind around a portion of the wheel when the patient exhales, and to unwind when the patient inhales; and
a processor configured to detect an angular position of the wheel and to present the patient with a corresponding feedback signal.

2. The device of claim 1, further comprising a transducer configured to convert the angular position of the wheel into the feedback signal.

3. The device of claim 2, wherein the transducer comprises an accelerometer.

4. The device of claim 2, wherein the transducer comprises at least one of a strain gauge, rotary encoder, gyrometer, and magnetometer.

5. The device of claim 2, wherein the transducer comprises a slotted wheel.

6. The device of claim 5, further comprising an emitter and a detector configured to pass a beam through the slotted wheel.

7. The device of claim 6, wherein the processor is configured to track successive interruptions in the beam.

8. The device of claim 1, wherein the feedback signal comprises a visual display.

9. The device of claim 1, wherein the feedback signal comprises a haptic signal.

10. The device of claim 1, wherein the feedback signal comprises a visual signal combined with one of a haptic signal and an audio signal.

11. A method of facilitating a breath-hold event at a predetermined point within a respiratory cycle, comprising:
providing a strap configured to wrap around a patient and having a segment configured to wind around a portion of a spring-biased wheel when the patient exhales, and to unwind when the patient inhales;
providing a processor configured to detect an angular position of the wheel; and
presenting the patient with a feedback signal corresponding to the angular wheel position.

12. The method of claim ii, further comprising:
converting the angular position of the wheel into the feedback signal using a transducer.

13. The method of claim 12, wherein the transducer comprises an accelerometer.

14. The method of claim 12, wherein the transducer comprises at least one of a strain gauge and a rotary encoder.

15. The method of claim 12, wherein the transducer comprises at least one of a gyrometer and a magnetometer

16. The method of claim 12, wherein the transducer comprises a slotted wheel.

17. The method of claim 16, wherein the transducer further comprises an emitter and a detector configured to pass a beam through the slotted wheel, and tracking successive interruptions in the beam.

18. The method of claim ii, wherein presenting the feedback signal comprises displaying at least one of a graphical, audible, and haptic indication of a breath-hold set point.

19. The method of claim 18, further comprising increasing the amplitude of the feedback signal as the set point is approached.

20. A respiratory set point tracking device, comprising:
a belt configured to extend around a patient and engage a spring-biased wheel; and
a transducer configured to convert an angular position of the wheel into a haptic feedback signal.
